(19) United States
(12) Patent Application Publication
(10) Pub. No.: US 2010/0247638 A1
(43) Pub. Date: Sep. 30, 2010

(54) ORGANOLEPTICALLY IMPROVED DIETARY FIBER COMPOSITION AND A PROCESS THEREOF (TEESTAR)

(75) Inventors: Villoo Morawala Patell, Karnataka (IN); Jagadeesh Badamaranahalli Henjarappa, SR., Karnataka (IN); Kamalendra Singh, Karnataka (IN); Chandramohan Bangalore Siddaiah, Karnataka (IN)

Correspondence Address: FENWICK & WEST LLP, SILICON VALLEY CENTER, 801 CALIFORNIA STREET, MOUNTAIN VIEW, CA, 94041, US

(73) Assignee: AVESTHAGEN LIMITED, Bangalore, Karnataka (IN)

(21) Appl. No.: 12/740,328

(22) PCT Filed: Dec. 11, 2007
(86) PCT No.: PCT/IN07/00580

§ 371 Date: Apr. 28, 2010

(30) Foreign Application Priority Data
Oct. 29, 2007 (IN) 2450/CHE/2007

(51) Int. Cl. A61K 9/64; A23L 1/212; A23F 5/22; A61K 38/00; A61P 3/10

(52) U.S. Cl. 424/456; 426/102; 426/615; 426/424; 514/6.9

(57) ABSTRACT

The present invention is in relation to an organoleptically improved dietary fiber composition from Trigonella foenum-graecum. In addition, the present invention provides a process to obtain an organoleptically improved dietary fiber composition comprising protein and galactomannans for treatment of diabetes.

### FIELD OF INVENTION

The present invention is in relation to the process of extraction of the fenugreek seeds and the extract thereof. More particularly the invention relates to a process for improving the organoleptic property of the dietary fiber obtained from the seeds of fenugreek by debitterisation, deodourisation and decolourisation of the seed. The invention further relates to fiber supplement that are non-toxic, effective and easily administrable. The invention subsequently relates to the use of the extracts as a supplement or a medicament useful in the management of disease or conditions characterized by increased blood sugar levels comprising the administration of a therapeutically effective amount of the composition containing the extract.

### BACKGROUND OF INVENTION

The herb Trigonella foenum-graecum, popularly known as fenugreek, belongs to family Leguminosae and has been in use since time immemorial by practitioners of Ayurvedic and Traditional Chinese Medicine. It is a commonly eaten food and a spice in many parts of the world. In India, the leaves and seeds are used as vegetables and the ripe seed further, finds numerous applications in the traditional systems of medicine. Fenugreek seeds are known to help in digestion of food and as an emollient it is used in poultices for boils, cysts and other complaints. Traditionally it has been used for treating various conditions like colic flatulence, dysentery, diarrhea, dyspepsia with loss of appetite, chronic cough, dropsy; enlargement of liver and spleen, rickets, gout and diabetes. Its use in post-natal care and as a galactagogue for nursing mothers and milch cattle to increase lactation is also well known. The seed has several applications in veterinary medicine as well. Modern medicine has also now begun to acknowledge the traditional medicinal applications of fenugreek seeds and provide scientific validation for many such uses. It has been established that the saponins of trigonella and the galactomannans a polysaccharide present in the seed albumen, have several beneficial effects including glucose and cholesterol metabolism.

Besides the aforesaid medicinal uses, it is also said to have preservative activity and hence are added to pickles, chutneys and other similar products. The seeds or the extract are used in bakery products, frozen dairy products, meat products, relish, condiments, candy, gravy sauces, gelatin puddings and in alcoholic and non-alcoholic beverages.

The seeds are the officinal part and are essentially dicotyledonous in nature consisting of two whitish translucent endosperm halves enclosed by a wrinkled brown-yellow seed coat or the husk. Between the two endosperm halves is sandwiched, a yellowish germ portion, which is mainly composed of good quality of edible protein, besides ether extractable, which are 7-9% fatty oil and flavoring essential oil. Fibrous material, mainly from the husk, contains insoluble fiber mainly comprising of cellulose, lignin and some hemicelluloses, while the endosperm contains the soluble galactomannans gum along with other pectin's and some hemicelluloses. Endosperm galactomannans, which accounts for nearly 50% of dry seed, is the reserve seed polysaccharides that are used up during the germination and growth of the plant embryo, till it starts photosynthesis. All galactomannans gums have a strong, but variable tendency to bind and hold water. Both the soluble and insoluble fibers constitute the dietary fibers of trigonella which is being exploited commercially for various kinds of applications. These dietary fibers are the non-absorbable and indigestible fibrous portion, which is not assimilated by the body and is non-caloric and has substantially no nutrition value. The dietary fibers are known to exert lot of beneficial effects on human health. There is ample evidence suggesting that trigonella is being utilized to normalize the rise in blood glucose level in conjunction with insulin and also known for its hypotensive and hypocholesterolemic effect. It has been confirmed by animal experiments and clinical tests on humans that ingesting the food compounded with fenugreek gum lower the blood sugar level and blood lipid levels and that these effects are due to the presence of galactomannan polysaccharide gum—a soluble dietary fiber present in seed endosperm/albumen. Fenugreek seeds may slow gastric transit time. It thickens the ingested food to form a gel in stomach trapping fat, sugars and starch hydrolyzing, amylase enzymes to slow down sugar absorption. The gel, which appears like ‘fat’ inside the body, signals the gall bladder to empty bile into the stomach. The gel then irreversibly traps lipid-emulsifying bile salts and prevents their reabsorption. Thus, emulsification and absorption of lipids including cholesterol results in lowering of blood lipid. This in turn reduces hypertension and chance of heart attack. Slowing the rate and extent of glucose absorption in the gut is not likely to be the sole mechanism for hypoglycemic benefit. An amino acid present in fenugreek seeds (4 Hydroxyisoleucine) that increases glucose-induced insulin release has been isolated and has been shown to be anti hyperglycemic. Fenugreek seeds contain the alkaloid trigonelline, which has a hypoglycemic effect. In view of the potential health benefits that the dietary fibers derived from trigonella seeds are known to exert particularly in respect to diabetes, increased attention is being given to the development of the method of extraction and purification of this polysaccharide.

Diabetes mellitus is one of the most prevalent degenerative diseases, which is generally characterized by the abnormalities in the body's ability to use sugar. Mainly three forms namely Type 1 diabetes, Type 2 diabetes and Gestational diabetes are known to occur. However, the diabetes epidemic relates particularly to type 2 diabetes and is largely discussed here in the context of trigonella.

Diabetes type 1 is an autoimmune disease that occurs when the body's immune system turns against a part of the body. In diabetes, the immune system attacks the insulin-producing beta cells in the pancreas and destroys them resulting in production of very little or no insulin. Therefore, a person afflicted by type 1 diabetes is dependent on daily dose of insulin. At present, scientists however have not been able to underpin the exact causes for the body's immune system to attack the beta cells, but they believe that autoimmune genetic, and environmental factors are involved. It develops most often in children and young adults, but can appear at any age. The gestational diabetes is however largely observed in pregnant women and is mainly characterized by temporary imbalance of the blood sugar level.

Type 2 diabetes is the most common form of diabetes afflicting approximately 90-95% of the people. This form of diabetes is generally associated with older age, obesity, family history, previous history of gestational diabetes, physical inactivity and ethnicity. About 80% of people with type 2 diabetes are overweight. It is increasingly being diagnosed in children and adolescents. In type 2 diabetes, the pancreases usually produce enough insulin, but the body is unable to use the insulin effectively, resulting in a condition called insulin resistance. After several years, insulin production decreases and the result is the same as for type 1 diabetes i.e., glucose builds up in the blood and the body cannot make efficient use of its main source of fuel. In the long term, diabetes also leads to other related problems like atherosclerosis, hyperlipidemia, retinal damage, neurological damage, and blindness due to spikes in blood sugar in patients during the day.

Diabetes chiefly a life style disorder is incurable. Presently, this disorder is managed by taking popular drugs available in the marketplace, which is largely based on blood sugar management mechanisms and through careful diet regime. These drug based therapies can be broadly categorized into three viz., pancreatic stimulators, insulin sensitizers and insulin. The Pancreatic stimulators help to increase secretion of insulin by stimulating the pancreas and primarily address the diabetes caused by inadequate insulin secretion. The Insulin sensitizers improve the cell's sensitivity to the presence of insulin, thereby improving the uptake of glucose into the cells, leading to better blood sugar while the Insulin is administered exogenously in the case of people suffering from both type I and type II diabetes.

Much of the available synthetic drugs are associated with several side effects caused due to long duration usage and since diabetes is a chronic, long-duration disease, these drugs need to be taken on a continuous basis. Hence, the risks of taking these medicines increase several folds thereby aggravating the problem and leading to other related complications. There are reports of hypoglycemia as a side effect due to persistent intake of Sulphonylureas. Similarly, Biguanides are known to cause lactic acidosis. Oral hypoglycemia drugs are also known to cause GIT irritation, weight gain, hypertension, etc. On continuous and constant exertion, the diabetic person is also vulnerable to pancreatic fatigue. In addition, it is also seen that many of the current existing drugs lead to drug resistance in patients with long durations of use. Therefore, the needs for safe, effective and patient compliant treatment with minimal side effects are imperative.

Consequently, a large number of botanicals are being explored in order to develop safe and efficacious drugs that can help in the management of blood sugar in diabetes mellitus patients. These plants based drugs are considered to be relatively much safer for long-term usage as they are known to cause minimal side effects and drug resistance. The treatment of diabetes is a particularly promising area for botanicals as most botanicals derive their effectiveness from a mixture of active molecules, acting in concert. Multiple agents attacking multiple targets simultaneously present decided advantages over conventional drugs, which are each based on one compound that produces one action. The application of Trigonella foenum-graecum extract in countering hyperglycemia and reducing cholesterol or triglyceride in plasma renders the species, as one of the promising candidates useful for developing therapeutics for the management of these conditions.

Considerable attention is being given to develop effective antidiabetic therapies based on trigonella, its standardization as a drug, its mode of administration, improvement of efficacy and organoleptic properties. As per the ancient Indian practice of Ayurveda and Naturopathy, fenugreek seed is traditionally taken in a powdered form, or boiled with water, or as a sprouted seed for the control of blood sugar. The varieties used for medicinal purposes have smaller grains, are dark brown in color and bitter in taste when compared to the varieties used for culinary purposes. Also, it appears that the medicinal effect of control of blood sugar obtained with the consumption of fenugreek seed is widely varying and cannot be relied upon as a reliable agent for control of blood sugar. Hence, there is a need to overcome all these limitations yet retain the beneficial properties for which trigonella seeds are well known.

Given the usefulness of the dietary fiber derived from trigonella, enormous effort has been directed towards isolation and fractionation of dietary fibers from trigonella. However, Trigonella fiber extract known in the art have unpleasant taste, smell and colour which makes them less patients compliant to such an extent that the bitterness factor disallows ingestion by oral administration. The bitterness present in conventional extracts of fenugreek can be attributed to the presence of the hydrolytic breakdown of lipids present in the fixed oil fraction. The problem is closely associated with the use of conventional processes that employ steam or water extraction of components from fenugreek seeds. The hydrolysis of lipids, which occurs over a period of time leads to rancidity and imparts the undesirable taste, the characteristic colour to the seed. The bitterness and darkened coloration is thereon passed on to the residual dietary fiber of fenugreek even after the extraction. A dietary fiber product having such bitterness and coloration is not easily acceptable as a food product, supplement or additive. Efforts have been made to remove these components which are responsible for unpalatable taste, colour and odour and isolate the useful components by employing different processes.

Fenugreek gum purified by employing the method disclosed herein yields a completely odorless, tasteless and whitish substance. The method disclosed herein yields gum in the range of 35 to 75%, which is far superior compared to the other processes. The dietary fiber or the purified fenugreek gum isolated as per this method can be particularly recommended to those, who are unable to take whole seed powder due to its odor and bitter taste. These fibers can also be used to make formulations, pharmaceutical compositions or functional food which could serve as a source of highly concentrated dietary fiber in a food supplement.

### PRIOR ART

The particular focus on isolation of soluble dietary fiber can be attributed to its physiological and biochemical activity, which makes them useful in the treatment of conditions relating to diabetes, obesity and cardiovascular diseases.

The prior art processes of isolation, as reviewed below are based primarily on organic solvents, which may leave toxic residues and may pose problems during large scale handling. Moreover, the time and technique intensive processes are cumbersome and costly at the same time are unable to result in desired quality dietary fibers. For instance, U.S. Pat. No. 5,847,109 of 1998 entitled “Galactomannan products of compositions containing the same by Garti et al describes a process of isolation involving use of organic solvents and high temperatures as well as specialized equipment for isolation of various components from the trigonella seed. Dietary fibers are also extracted using the same equipment and the same seeds following numerous treatments with different solvents. The process also involves treatment with polar alcohols, which may reduce the yield of the dietary fiber. The process yield dietary fibers with high protein content and to further reduce the protein content to chromatographic techniques are suggested which are difficult and expensive.

It also claims use of Fenugreek Galactomannan as a nutraceuticals in reducing post-prandial glucose, insulin response & cholesterol levels. They have quoted results of a one-week study on 22 subjects for this claim. The quantity of fenugreek derived galactomannan administration to them was 10 grams.

Similarly, Rao. G. B. et al U.S. Pat. No. 6,495,175, in one of the embodiments discloses a process of flaking, an essential step for separation of seed coat from cotyledon which is difficult to be carried out at ambient moisture content of fenugreek seeds. This makes the isolation process time intensive since tempering requires initially determination of the moisture content of the seed, followed by addition of a fixed quantity of water to achieve the desired moisture level uniformly. This not only increases the time of extraction process but also some times lead to hydrolysis of the lipids leading to discoloration of the final product. In another embodiment of the same patent fenugreek seeds are treated with polar alcohols at high temperatures to isolate the oleoresin component prior to isolation of soluble dietary fiber; however the process may lead to loss of some amount of dietary fibers. Dietary fibers are also further isolated by heating the fenugreek flakes with water at 60 degrees for 4 hrs which may affect the quality of the final product as well as the viscosity of the dietary fiber obtained.

Rao G. B. et al in U.S. Pat. No. 6,495,175 and US patent application 20010024665A1 describes a two step solvent extraction process wherein first extraction isolates fixed oils and second extraction isolates oleoresins leaving the light yellow to light brown dietary fiber.

The invention also describes a special extraction system for better efficiency. As known in the prior art increasing surface area of the material to be extracted improves the extraction efficiency, hence the process of invention utilizes grinding and sieving of the fenugreek seeds for imparting higher surface area for better extraction.

Use of high temperature, solvents and specialized extractor makes the process very difficult on the commercial scale. Since the embryo is not separated from the dietary fiber at the time of grinding or isolation the fibers obtained have higher protein content, which is not a desirable attribute. There is also no mention of any diabetic treatment with any of these compounds.

U.S. Pat. No. 5,997,877 of 1999 by Peter Chang discloses a process for isolation of oleoresins, saponins and a soluble dietary fiber. The process comprises of tempering fenugreek seeds to moisture level of 16-22%, followed by flaking using roller mill and sieving to separate the seed coat from cotyledon. The separated seed coat portion is treated with hot water for several hours, centrifuged and precipitated using a polar alcohol. Precipitate thus obtained is rich in soluble dietary fiber, which is further washed and dried. It also describes a process for the fractionation of Fenugreek seeds & extraction of the various fractions such as soluble dietary fibre, deflavoured fenugreek seed, high protein fenugreek meal, etc. Tempering/flaking before extraction is carried out to increase the extraction recovery ratio and decrease the contact times. To achieve proper tempering seeds are mixed with water and kept aside for 24 hrs prior to flaking. The product so obtained is rich in soluble dietary fibers. There is no reference to any diabetic treatment.

The process listed in U.S. Pat. No. 5,658,571 by Gopalan et al also involves a special kind of reactor and solvents and has similar disadvantages.

Osband M. E. describes another solvent-based extraction process in PCT application WO9925197. The process results in a product having a high amount of protein (about 20-40%) which not only reduces the amount of dietary-fiber content in the end product but also is undesirable for it contributes to the unpleasant brown coloration.

PCT application WO/0174371A1 by Bourret E., disclosed a method of using organic solvents to obtain Trigonella foenum-graecum mucilage in the form of flour with grain size distribution less than 100 um, consisting mannose, galactose, glucose, arabinose, xylose, rhamnose, D-galacturonic acid, galactomannans, and proteins. The key feature of the invention is pulverization of non-lipid fraction of fenugreek seed at sub zero temperatures (−195 degrees) to improve the solubility of non-lipid fraction of fenugreek seed in the extracting solvent. This requires a specialized facility to carry out such an operation thereby increases the cost of isolation of the dietary fiber.

PCT application WO 0128673A1 describes the manufacture or isolation of galactomannans using various organic solvents, and the use of such galactomannans.

Mechanical process of isolation has also been described in the Japanese patent application JP 2001025265. The process involves special equipment comprising of a cylindrical vessel made up of metal net with 150 mesh screen and rotary wings for separating the endosperm. This process needs specialized equipment, moreover the husk and the seed of fenugreek are very difficult to pulverize and therefore keeping a 150-mesh screen means pulverization for a prolonged period leading to generation of local hot spots which may affect the properties of the final product.

Although patents such as U.S. Pat. No. 5,288,618 by Hardin, U.S. Pat. No. 6,039,980 by Baichwal and U.S. Pat. No. 6,063,402 by Gebert describe pharmaceutical applications of galactomannan, none of these applications are focused on improving the organoleptic properties.

The Application WO/2005/049211 (Pilgaonkar et al.) teaches the method of isolation of an insoluble fiber rich fraction from Trigonella foenum-graecum seeds. The multifunctional fiber rich fraction (FRF) and highly purified FRF are useful as excipients for pharmaceutical dosage forms for various routes of administration.

Prior art review shows attempts made to isolate the dietary fibers in higher purity are largely unsuccessful owing to non-specific isolation techniques, use of costly and specialized equipments, use of organic solvents for extraction and energy intensive methods. Some of them also raise safety issues. The prior art fails to reveal processes targeted towards minimizing the steps and costs involved in the isolation of soluble dietary fibers with improved organoleptic properties but retaining the same beneficial effects that can be used as a pharmaceutical composition/food supplement. The art also fails to teach a process of isolation of a fraction of dietary fiber of fenugreek seed with an yield of soluble dietary fiber ranging greater than 35-75% with a protein content not more than 30%.

A review of prior art shows that the method for the isolation of soluble dietary fiber of trigonella seeds through the means of organic solvents is well known in the art. However, most of these extracts are not well received commercially owing to the unpleasant taste and appearance of the final product at the time of consumption. The present invention alleviates these problems pertaining to taste, odour and appearance and hence is likely to be received well by consumers. The number of steps and cost involved in the isolation of dietary fibers is less compared to the invention disclosed in the prior art cited herein. The present inventors have found that the process of instant invention leads to a novel fraction of soluble dietary fibers resulting in a product rich in galactomannan and with organoleptic properties far superior than the products already available in the market.

None of the above patents, taken either singly or in combination, is seen to describe the instant invention as claimed. Thus, obtaining organoleptically improved dietary fiber composition from Trigonella foenum-graecum using the process of instant invention will therefore helps in addressing the problems associated with the prior art. It is the sequence of steps involved which are unique and which has resulted in arriving at organoleptically improved dietary fiber composition for management of diabetes and its related disorders. Accordingly, the product of the instant invention has been named as “TEESTAR”.

### OBJECTS OF THE PRESENT INVENTION

The principal object of the present invention is to develop an organoleptically improved dietary fiber composition from Trigonella foenum-graecum.

Another object of the present invention is to provide a process for extraction of organoleptically improved dietary fiber composition from Trigonella foenum-graecum. Yet another object of the present invention is to make use of organoleptically improved dietary fiber composition from Trigonella foenum-graecum for treating of diabetes. Still another object of the present invention is to provide a method to fractionate trigonella seeds and recover substantially pure and useful fraction of extracts of fenugreek seeds using a commercially feasible processing method to maximize the total value of the fractionated products from the process.

Still another object of the present invention to provide soluble fiber composition and methods for fractionation and isolation of bioactive compounds from fenugreek seeds that involve efficient but less number of steps and which is economical to produce.

Still another object of the present invention to provide soluble fiber compositions and methods for the extraction and separation of bioactive compounds from fenugreek seeds, which provides a high quality and quantity extract yield.

Still another object of the present invention to provide novel compositions and methods for the extraction and separation of bioactive compounds from fenugreek seeds which are capable of aiding in management of blood glucose levels.

### STATEMENT OF INVENTION

Accordingly, the present invention provides an organoleptically improved dietary fiber composition obtained from Trigonella foenum-graecum for management of diabetes, said composition comprising proteins 20% w/w to 30% w/w, and galactomannans 32% w/w to 80% w/w optionally along with acceptable additives; a method of treating diabetes in a subject in need thereof, said method comprising step of administering acceptable amount of organoleptically improved dietary fiber composition obtained from Trigonella foenum-graecum, said composition comprising proteins 20% w/w to 30% w/w, and galactomannans 32% w/w to 80% w/w optionally along with acceptable additives to the subject; and a process for extraction of organoleptically improved dietary fiber composition from plant Trigonella foenum-graecum comprising proteins 20% w/w to 30% w/w, and galactomannans 32% w/w to 80% w/w, optionally along with acceptable additives, said process consisting steps of soaking the plant parts overnight in a solvent, powdering plant parts to obtain powder, extracting the powdered plant parts using a solvent, separating the extracted components, precipitating the separated components to obtain gum and washing precipitated gum followed by drying to obtain organoleptically improved dietary fiber.

### BRIEF DESCRIPTION OF THE ACCOMPANYING DRAWINGS

The following drawings are part of the present specification and are included to further demonstrate certain aspects of the invention. The invention may be better understood by reference to one or more of these drawings in combination with the detailed description of the specific embodiments presented herein.

FIG. 1: Galactomannan Content in various samples

FIG. 2: Estimation of protein content in various samples

FIG. 3: Changes in the post-prandial glucose levels

FIG. 4: Changes in the HbA1c levels

FIG. 5: Color comparison of the product of instant invention vis-à-vis the organoleptically not improved product

### DETAILED DESCRIPTION OF THE PRESENT INVENTION

The present invention is in relation to an organoleptically improved dietary fiber composition obtained from Trigonella foenum-graecum for management of diabetes, said composition comprising proteins 20% w/w to 30% w/w, and galactomannans 32% w/w to 80% w/w optionally along with acceptable additives.

In another embodiment of the present invention wherein said composition preferably comprises proteins 25% w/w, and galactomannans 75% w/w.

Yet another embodiment of the present invention, wherein said dietary fiber is extracted preferably from the seeds of Trigonella foenum-graecum.

Still another embodiment of the present invention, wherein said organoleptic characters include decolorization, deodorization and debitterization.

Still another embodiment of the present invention, wherein the fiber is a soluble food supplement.

Still another embodiment of the present invention, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

Still another embodiment of the present invention, wherein said composition is formulated into dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs. The present invention is in relation to a method of treating diabetes in a subject in need thereof, said method comprising step of administering pharmaceutically acceptable amount of organoleptically improved dietary fiber composition obtained from Trigonella foenum-graecum, said composition comprising proteins 20% w/w to 30% w/w, and galactomannans 32% w/w to 80% w/w optionally along with acceptable additives to the subject.

In another embodiment of the present invention, wherein the subject is an animal or human being.

Yet another embodiment of the present invention, wherein the soluble dietary fiber from Trigonella foenum-graecum, is administered at dosage ranging between 30 to 125 mg/kg body weight.

Still another embodiment of the present invention, wherein the soluble dietary fiber from Trigonella foenum-graecum, is administered preferably at dose of about 50 mg/kg body weight.

Still another embodiment of the present invention, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

Still another embodiment of the present invention, wherein the soluble dietary fiber from Trigonella foenum-graecum, is formulated into dosage forms selected from a group comprising tablet, troches, lozenges, aqueous or oily suspensions, dispersible powders or granules, emulsion in hard or soft gel capsules, syrups, elixirs, phyotceuticals and neutraceuticals.

Still another embodiment of the present invention, wherein the soluble dietary fiber from Trigonella foenum-graecum, is non-toxic and free of adverse effects.

The present invention is in relation to a process for extraction of organoleptically improved dietary fiber composition from plant Trigonella foenum-graecum comprising proteins 20% w/w to 30% w/w, and galactomannans 32% w/w to 80% w/w, optionally along with acceptable additives, said process consisting steps of:
(a) soaking the plant parts overnight in a solvent;
(b) powdering plant parts to obtain powder;
(c) extracting the powdered plant parts using a solvent;
(d) separating the extracted components;
(e) precipitating the separated components to obtain gum; and
(f) washing precipitated gum followed by drying to obtain organoleptically improved dietary fiber.

In another embodiment of the present invention, wherein the plant parts preferably seeds were soaked in water over night followed by draining the brown colored water 3 to 4 times.

Yet another embodiment of the present invention, wherein said dietary fiber is milky white in color, tasteless and flavorless.

Still another embodiment of the present invention, wherein the plant parts are selected form a group comprising root, shoot, leaf and seeds or the whole plant.

Still another embodiment of the present invention, wherein the preferred plant parts are seeds.

Still another embodiment of the present invention, wherein the plant parts are powdered either manually or mechanically.

Still another embodiment of the present invention, wherein the solvents are selected from a group comprising water, concentrated hydrochloric acid and combinations thereof.

Still another embodiment of the present invention, wherein the solvents are preferably combination of water and hydrochloric acid.

Still another embodiment of the present invention, wherein the water to acid ratio is ranging from 1:100 to 1:1000.

Still another embodiment of the present invention, wherein the water to acid ratio is preferably 1:200.

Still another embodiment of the present invention, wherein the extraction is carried out for a time period ranging from 3 to 16 hrs.

Still another embodiment of the present invention, wherein the extraction is carried out for a time period preferably about 4 hrs.

Still another embodiment of the present invention, wherein said extracted components are separated by a method selected from a group comprising centrifugation, filtration and sedimentation.

Still another embodiment of the present invention, wherein said precipitation is achieved using organic solvents selected from a group comprising methanol, ethanol, anhydrous ethanol, propanol, butanol, ethyl acetate and combinations thereof.

Still another embodiment of the present invention, wherein said precipitation is achieved preferably using solvents ethyl alcohol and ethyl acetate at a ratio of 2:1.

Still another embodiment of the present invention, wherein said precipitation results in obtaining a gum as heavier phase.

Still another embodiment of the present invention, wherein said gum is washed using a solvent selected from a group comprising water, methanol, ethanol, anhydrous ethanol, propanol, butanol and combinations thereof.

Still another embodiment of the present invention, wherein said gum is washed using hydro alcoholic mixture at a concentration ranging from 80% to 95%.

Still another embodiment of the present invention, wherein said gum is washed using anhydrous alcohol at a concentration preferably about 95% Still another embodiment of the present invention, wherein said gum is washed for a time period ranging from 30 to 90 minutes.

Still another embodiment of the present invention, wherein said gum is washed for a time period preferably about 35-45 minutes.

Still another embodiment of the present invention, wherein the gum is dried by freeze drying method at a temperature preferably about −70° C.

Still another embodiment of the present invention, wherein the dietary fiber is obtained from Trigonella foenum-graecum having a yield ranging from 35% to 80%.

Still another embodiment of the present invention, wherein the dietary fiber is obtained from Trigonella foenum-graecum, having a yield preferably of about 78%±2%.

It will be readily understood that the components of the present invention, as generally described herein, could be arranged and designed in a wide variety of different configurations. Thus, the following more detailed description of the embodiments of the system and method of the present invention is not intended to limit the scope of the invention, as claimed, but is merely representative of the presently preferred embodiments of the invention.

The term “dietary fiber” here refers to soluble and insoluble fiber and covers hemicellulose, cellulose, mucilage, lignin and optionally resistant starch and other materials like proteins and vitamins and minerals of fenugreek seed. It also contains the non-absorbable, indigestible fibrous portion of the food. It is not assimilated by the body and is non-caloric and has substantially no nutrition value. Insoluble dietary fiber mainly comprises cellulose, lignins and some hemicelluloses; while, soluble dietary fiber includes but is not restricted to pectin, gums and some hemicelluloses. The fenugreek dietary fiber of the invention comprises substantially all of the insolubles originally contained in the seed. As regards the soluble fiber, a small part of it contained in the seed is extracted out by the acidic solvent in the process of the invention. The extracted out soluble fiber is a small part of the mucilage (gum) of the seed. Substantially all of the proteins also remain in the residue fiber and so also the vitamins and minerals. The residue also contains any other unextracted or partly extracted constituent of the seed. The actual benefit provided by soluble fiber depends upon its content, the source from which it was isolated and the process by which it was isolated.

As used herein, “anti-diabetic” or “hypoglycemic” or “antihyperglycaemic” compound or composition generally refers to an agent that lowers blood glucose levels. As a general guideline, without being limited to the values suggested herein, if blood glucose level is decreased by at least about 100 mg/dl, then the compound is considered to be a hypoglycemic agent. Such an agent or any other agent that may lower blood glucose levels to other accepted standards of hypoglycemic effect may be used to treat diabetes or to prevent the incidence of diabetes. The hypoglycemic or anti-diabetic effect can be measured by a variety of methods including, but not limited to, measuring the blood glucose levels, the rate of insulin binding to its receptor, the level of insulin secretion from pancreatic beta cells, and inhibition of glucohydrolase activity.

As used herein, “extract” refers to a concentrated preparation of the essential constituents of the medicinal plant. Typically, an extract is prepared by drying and subsequently grinding the dried material. The extraction process may then be performed with the help of an appropriate choice of solvent. The extract may be used in liquid form, or it may be mixed with other liquid or solid medicinal herbal extracts. The extract may then be further evaporated and thus concentrated to yield a soft extract and/or eventually a dried extract, by means of spray drying, vacuum oven drying, fluid-bed drying or freeze-drying. Alternatively, the medicinal herbal extract may be obtained by further precipitating solid extracts from the liquid form.

In this specification, the term “extraction” refers, depending on the context, to either the unit operation of extraction wherein a liquid solvent is brought into contact with a solid material with the object of dissolving (leaching out) one or more components thereof, or a process having the extraction operation as one of the steps thereof. During liquid-solid extraction, an extraction solvent is brought into contact with a solid material that contains a solute to be extracted there from. During the extraction, the solute is dissolved in the extraction solvent. The concentration of the solute in the solvent increases as the extraction proceeds till the solvent is saturated with the solute or all of the solute contained in the solid material is extracted out, whichever comes first.

An extraction may be carried out at substantially the boiling point of the solvent. Generally, the extraction is carried out at any temperature from above ambient to substantially the boiling point of the solvent. The extraction can also be carried out at substantially the condensing temperature (the temperature at which the solvent condenses in the condenser of the apparatus) of the solvent but extractions at other temperatures are within the scope of the invention. Extraction at the highest feasible temperature, offers a high leaching rate and consequently a low contact time. In specific embodiments, the operating temperatures are room temperatures.

In this specification, a solvent comprising the solute in solution is referred to as the “solution” or as the “extract” and the solid material after extraction is referred to as the “extracted solids” or “extracted mass”. Sometimes, the “solution” is also referred to merely as the “solvent” particularly where the solute content therein is fairly small. In case of there being two stages of extraction, the terms used are “first extraction solvent” or “first solvent”, “first solid material”, “first extract” and “first extracted solids” etc., for the first stage and similarly for the second stage. Where a particular fraction of a spice such as fixed oils or oleoresin is being extracted, the selected solvent would dissolve several, or even all of the individual components (compounds) which constitute the respective fraction. References to a solute herein below, generally refers to the component(s) that is (are) extractable by the respective extraction solvent.

There are a large number of solvents (or mixtures thereof) that may be used as extraction solvents. In one embodiment, the solvent for the first stage is water with conc. HCl and for the second stage is 90-95% aqueous ethanol. Any aqueous ethanol of 90% and above, or preferably, 90-95% strength may be used. A solvent mixture can comprise any two or more different solvents. The second stage solvent ethanol generally extracts out the saponin content along with the oleoresin and fixed oils.

The extraction parameters that affect the degree and rate of extraction include: 1) the contact ratio, which is the ratio of the amounts of solvent and solids contacted; 2) the extraction temperature, which is the temperature at which the contacted mass of solvent and solids is maintained during extraction; 3) the extraction or contact time, which is the period during which the solvent and solid are contacted; 4) rate of removal of the extract from the extracted mass; 5) the particle size of the solids being treated by extraction; and 6) the solvent concentration.

The raw material for the process of the invention is fenugreek seeds. The seeds may be virgin, i.e., unextracted, or partly extracted, having undergone one or more previous extractions for the fractions, oleoresins, fixed oils, essential oil, dietary fiber or any of the other compounds that are constituents of the fenugreek seeds. The seeds may be given any preliminary treatment before extraction as necessary such as sorting, grading and cleaning. The seeds may be dried to moisture content as desired, prior to size reduction or extraction. The seeds may then undergo a size reduction operation comprising crushing, grinding, flaking, milling and/or screening as required. The seeds are generally crushed or ground before extraction so as to provide increased surface area for contact with solvent. Better contact between the solvent and solid results in a greater rate of extractions and reduces the contact time.

Conc. HCl is easy to remove from the first extract. The second solvent, such as ethanol, is also easy to remove by direct evaporation thereof or by other means. Traces of the second solvent, aqueous ethanol in the second extract even if remaining, do not pose any health hazard nor do they in any way distort the characteristics of the product. Generally, in the process of the invention, solvent traces are substantially completely eliminated, meaning that there may be very small traces of solvent remaining, but that the amounts are generally less than 20 ppm.

The contact ratio, that is, the ratio of the weight of the material to be extracted (kg) to the total volume (L) of solvent contacted therewith in either extraction stage generally ranges from 1:1 to about 1:10 and even higher. The contact ratios in one embodiment generally range from 1:1 to 1:5 for the first stage extraction and 1:1 to 1:7 for the second stage extraction.

The dietary fiber can be dried in any conventional dryers available in the art such as tray, vat, rotary, fluidized bed type or others. The dryer can have steam, electrical or other heating means to accelerate solvent removal. Trigonella extract is dried to stabilize them for storage or distribution. Drying always causes some loss of activity or other damage hence Lyophilization, also called freeze-drying, and is adopted herein which significantly reduces such damage. Lyophilization also helps to achieve a porous, friable structure. In one embodiment, the drying is conducted for about one hour to evaporate the solvent adhering to the solids and to reduce the moisture content to about 3%-5%.

The solvent is generally removed from the extracts by washing. Other methods of desolventising, i.e., of solvent removal and recovery, may be used and are considered within the scope of the invention.

When solvent is evaporated from the first extract, it yields substantially pure product which may then be sent for further operations as required, such as analysis, testing, packing and the like.

The fenugreek soluble dietary fiber was observed by the inventors to be suitable for edible purposes and its effectiveness has been established in tests. It is also rich in proteins and well provided with several vitamins and minerals. It is approximately light yellowish to whitish in color with little to no taste or odor and therefore forms an excellent substrate for taste, color and flavoring agents and can be easily blended. However, the dietary fiber may be fortified with further nutrients such as proteins, vitamins, minerals and others nutrients to form a fenugreek dietary fiber based product. The present invention is also directed to an herbal medicinal composition that can be administered to a person suffering from diabetes, preferably type II diabetes, which results in the lowering of the blood glucose level of the patient.

### Potential Plant

In one embodiment of the present invention, there is provided a novel process for preparing and recovering soluble fiber rich fraction from plant parts of Trigonella without the use of organic solvents or specialized equipments that comprises:
a. pulverizing selected plant material to a powder;
b. subjecting the powdered plant material to acid extraction;
c. lyophilizing the obtained extracts; and
d. analyzing the plant extracts for toxicity and presence of inhibitory activity against diabetes.

The choice of selected plant material may be of any type but is preferably selected from the seed or the fruit of the Trigonella plant.

The direct solvent extraction process involves extraction from plant parts in flasks at room temperature with polar and/or non-polar solvent(s). Typically, the extraction process is as outlined herein.

The solvents used in the case of direct extraction included acidic solvent alone and in combination with water.

In the embodiment of the invention, the extraction of soluble fiber is accomplished by extracting fenugreek seed material with a solvent at a room temperature for a period of contact time such that the seed components are absorbed by the solvent followed by separation of extracted seeds and the seeds components from the solvent. The solvent used in the preferred embodiment of the process is a mix of water and conc. HCl. Separating said solvent into heavy and light phases; precipitating the light phase with a precipitating agent to yield precipitated gum from the remainder of the light phase by solid/liquid separation; washing said precipitated gum and drying the same. Precipitation in the second embodiment is accomplished by mixing the light phase of the solvent with a precipitating agent such as, ethanol and ethyl acetate. The separation of the light phase of the precipitated solvent from the heavy phase thereof is accomplished by centrifugation or filtration, and the washing of the heavy phase, being precipitated gum, comprises mixing said heavy phase with a washing agent for a period of washing time, then separating the washed heavy phase from the washing agent (ethyl alcohol). The washing step might be carried out a second or numerous times to improve the cleanliness of the final product followed by the collection of the washed fibers and lyophilizing (at −70° C.) to yield the final product. Accordingly, the product obtained is named as “TEESTAR”.

In accordance with another aspect of the invention, separate in vitro tests are conducted to evaluate the toxicity of the extracts to ascertain the safe practical application of the said extracts. The bio therapeutic potential of the said extracts has been studied and confirmed through standard in vitro cell free and cell based assays.

Furthermore, also as readily apparent to one skilled in the art, the therapeutic compositions of the invention will need to meet certain criteria in order to be suitable for human or animal use and to meet the regulatory requirements. Thus, once the composition of the invention has been found to be suitable for animal administration, standard in-vivo and in vitro tests can be conducted to determine the information about the metabolism and pharmacokinetics of the compositions, including data on the drug-drug interactions where appropriate, which can be used to design human clinical trials. The present invention further contemplates that where toxicity is a factor, for example, in patients who cannot tolerate optimal or standard therapeutic dosages, or in cases where the patient's metabolism is compromised sub-optimal doses would be preferred. Also encompassed within the ambit of the invention is a pharmaceutical formulation suitable for use in the management of blood glucose level comprising extract as isolated from a Trigonella species, in admixture with a pharmaceutically acceptable carrier. The skilled addressee will appreciate that such compositions may comprise of plant extracts of the invention in any concentration, which is capable of giving rise to a therapeutic effect. Thus, therapeutic compositions can comprise plant extracts of Trigonella substantially devoid of undesirable contaminating compounds. The plant extracts may have, for example, undergone a number of solvent extraction steps substantially to separate out undesirable components from desirable components.

Whilst it is possible for the active extract to be administered alone, it is preferred to present the active extract in a pharmaceutical formulation. Formulations of the present invention, for medical use, comprise an extract of the invention together with one or more pharmaceutically acceptable carriers and optionally other therapeutic ingredients. The carrier(s) should be pharmaceutically acceptable in the sense of being compatible with the other ingredients of the formulation and substantially non-deleterious to the recipient thereof.

Formulations according to the present invention include those suitable for oral administration. The formulations may conveniently be presented in unit dosage form and may be prepared by any of the methods well known in the art of pharmacy. All methods include the step of bringing the active extracts(s) into association with a carrier which constitutes one or more accessory ingredients. In general, the formulations are prepared by uniformly and intimately bringing the active extract(s) into association with a liquid carrier or a finely divided solid carrier or both and then, if necessary, shaping the product into desired formulations.

Formulations of the present invention suitable for oral administration may be presented as discrete units such as capsules, sachets, tablets, lozenges, comprising the active ingredient in a flavored based, usually sucrose and acacia or tragacanth; pastilles comprising the active ingredient in an inert base such as gelatin and glycerin, or sucrose and acacia. Each formulation generally contains a predetermined amount of the active extract; as a powder or granules; or a solution or suspension in an aqueous or non-aqueous liquid such as syrup, an elixir, an emulsion or draught and the like.

In addition to the aforementioned ingredients, the formulations of this invention may further include one or more accessory ingredients selected from diluents, buffers, flavoring agents, binders, surface active agents, thickeners, lubricants, preservatives (including antioxidants) and the like.

Alternatively, the compositions are dietary supplements, food compositions or beverage compositions suitable for human consumption.

In a further aspect of the invention, the pharmaceutical formulations for preventing, treating, or managing diabetes and diabetes related disorders, comprise direct extracts of Trigonella. Such pharmaceutical formulations may contain a pharmaceutically acceptable carrier, excipients or diluents as outlined herein.

In a further aspect of the invention there is provided a comestible, that is to say, a foodstuff comprising at least an extract of the invention, typically in dried form, such as in a lyophilized form. Such foodstuffs may be used in a prophylactic manner and may contain further extracts having a similar function to the first added extract or further added extracts may be added that have a different prophylactic function. Thus a foodstuff could either comprise extracts that provide for a comestible having a single functional aspect, for example that of having a prophylactic effect against the occurrence of diabetes, or a comestible may have a multi-functional prophylactic effect against two or more disease types. It is thought that a similar multi-functional role could also be assigned to pharmaceutical formulations comprising two or more extracts possessing dissimilar therapeutic or prophylactic properties designed either for prophylaxis or for the treatment of more than one disease(s) in a mammal, particularly in a human.

The type of foodstuff or comestible to which at least an extract of the invention may be added includes any processed food such as confectionaries, baked products including breads such as loafs, and flat breads such as pitta bread, naan bread and the like, cakes, snack foods such as muesli bars, compressed dried fruit bars, biscuits, dairy products such as yoghurts, milk and milk-based products such as custards, cream, cheese, butter and creme fraiche, simulated dairy food products such as margarine, olive oil-based spreads, and low fat cream substitutes such as Elmlea products, fruit and vegetable juices, aerated drinks, such as carbonated soft drinks and non-aerated drinks such as squashes, Soya milk, rice milk and coconut milk and the like, pastas, noodles, vegetable, seed and nut oils, fruited oils such as sunflower oil, rapeseed oil, olive oil, walnut, hazelnut, and sesame seed oil and the like, and frozen confections such as ice creams, iced yoghurts and the like.

The term “nutraceutical” as used herein is now recognized in the art as referring to nutrition and food products, usually from natural sources, having some pharmaceutical benefits. With regard to the isolated galactomannans described hereinafter, it has been surprisingly found that they reduce post-prandial glucose and insulin response, as well as cholesterol levels.

The technology of the instant Application is further elaborated with the help of following examples. However, the examples should not be construed to limit the scope of the invention.

### Example: 1

The plant parts of Trigonella foenum-graecum, preferably the seeds were soaked in water overnight and draining the brown colored water 3 to 4 times before extraction. This helps in decolorizing of the water-soluble fibers. After extraction, the extract was treated with 0.1% HCl, centrifuged and the water-soluble fibers are precipitated using absolute alcohol in 1:2 proportions and the precipitate is dried under reduced vacuum and temperature.

### Example: 2

### Trigonella Aqueous Extract

1. Distilled waster (acidify with 750 μl of conc. HCl) was added to non-defatted Trigonella seed powder (the proportion of trigonella seeds to water is 1:30)
2. The mixture was stirred and centrifuged to remove the debris.
3. Precipitate the water soluble fibers using Ethyl alcohol and Ethyl acetate (2:1 ratio, i.e. 1.5 L Ethyl alcohol and 750 ml Ethyl acetate) to separate the fibers by filtering through cheesecloth
4. Wash the precipitate with ethanol and dry the precipitate preferably Freeze dry the precipitate under vacuum.

### Example: 3

### Ethanolic Extract of Trigonella

1. 100 ml of food grade ethanol was added to 10 g of trigonella seed powder
2. The mixture was stirred for 3 hr at room temperature and centrifuged at 3200 g for 20 minutes to remove the debris
3. The supernatant was collected and dried

### Example: 4

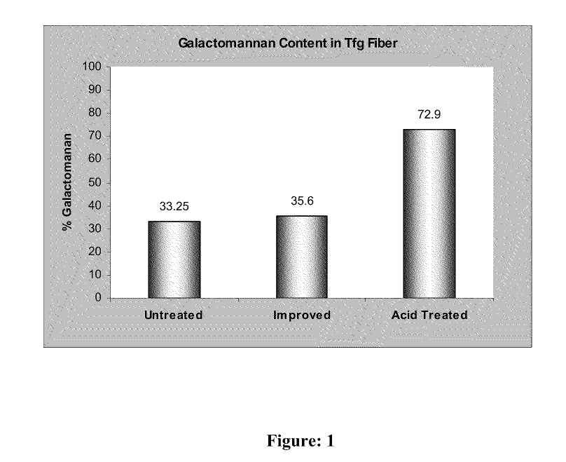

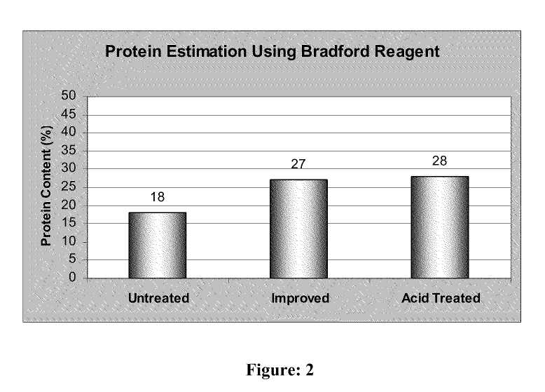

The product of the instant invention is characterized for the individual components present. Also, the product of instant invention is compared vis-à-vis the conventional, organoleptically not improved product. Accordingly, the below table provides the data to establish that the percentage of proteins and galactomannans were higher than the conventional, organoleptically not improved product. The galactomannans and proteins estimated are provided in FIG. 1 and FIG. 2. Also, the both the figures provide comparative analysis of the product of instant invention vis-à-vis the untreated and organoleptically unimproved products obtained from Trigonella foenum-graecum. Table: 1 provides the percentage values for proteins and galactomannans for the product of instant invention and organoleptically not improved product.

TABLE 1
Percentage of proteins and galactomannans in the product of instant
invention and its comparison with conventional product

| | AGT019Se08 (00) | |
|---|---|---|
| | Organoleptically not | AGT019Se08 (00) |
| | improved (or) Conventional | Organoleptically |
| Parameters | product | improved |
| Proteins (%) | 18 | 25 |
| Galactomannans | 32 | 75 |
| (%) | | |

### Example: 5

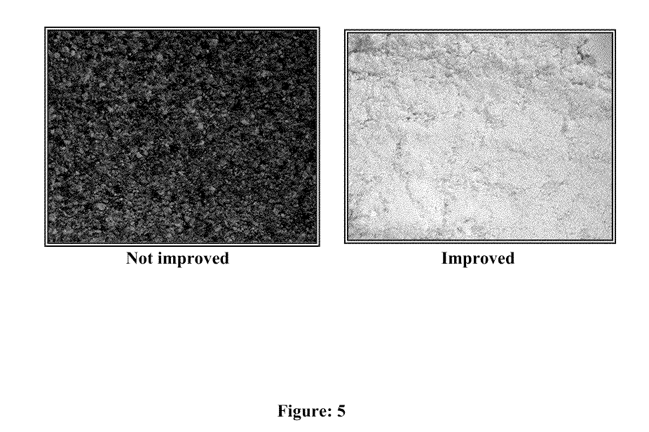

The organoleptic properties of the composition obtained by the process of instant invention are provided below. In addition the organoleptically improved product is compared with the product which is not improved organoleptically. In view of this it is evident that the product of the instant invention is associated with milky white color, tasteless and flavorless. Thus, the process of instant invention has indeed achieved a product which is organoleptically improved when compared with the conventional product. The comparison of the color of the product of instant invention vis-à-vis the conventional, organoleptically not improved product is shown in FIG. 5. Table: 2 provides the details regarding the color, odor and taste of the product of instant invention and its comparison vis-à-vis the conventional product.

TABLE 2
Organoleptic properties for the product of instant invention and its
comparison vis-à-vis the conventional product.

| | AGT019Se08 (00) | |
|---|---|---|
| | Organoleptically not | AGT019Se08 (00) |
| | improved (or) conventional | Organoleptically |
| Attributes | product | improved |
| Color | Muddy brown | Milky white |
| Taste | Characteristic taste | Tasteless |
| Flavor | Characteristic flavor | Flavorless |

### Example: 6

TABLE 3
Data to establish that the product of instant invention is indeed free of
toxicity and adverse effects

| | | | |
|---|---|---|---|
| Sl. No. | Parameters | Results | Test Method |

A Physical Parameters

| | | | |
|---|---|---|---|
| 1 | Density, g/cm³ | 0.99 | Client's Method |
| 2 | Moisture, % w/w | 9.3 | IS:2362:1973 |
| 3 | Moisture pick up, % w/w | 4.6 | IS:2362:1973 |
| 4 | Insoluble/Suspended | 92.47 | AOAC |
| | matter, % | | |

B Color

| | | | |
|---|---|---|---|
| 1 | Carotenoids, mg/100 g | Nil | IS:5886:1970 |
| 2 | Total Chlorophyl, mg/l | 0.04 | AOAC |

C Safety Parameters

| | | | |
|---|---|---|---|
| 1 | Lead, ppm | Not Detected | IS:12074:1987 |
| 2 | Copper, ppm | 18.23 | IS:11123:1984 |
| 3 | Arsenic, ppm | <0.1 | ICP:OES |
| 4 | Zinc, ppm | 79.97 | IS:9958:1981 |
| 5 | Cadmium, ppm | <0.1 | ICP:OES |
| 6 | Tin, ppm | <0.1 | ICP:OES |
| 7 | Hyperisine | Not Detected | HPLC |
| 8 | Agaric acid | Not Detected | HPLC |
| 9 | Hydrocyanic Acid, ppm | Not Detected | AOAC |
| 10 | Saffrole, ppm | Not Detected | GC |
| 11 | Aflatoxin B₁ | Not Detected | AOAC 17^{th} Edition |

D Microbiological Parameters

| | | | |
|---|---|---|---|
| 1 | Coliforms/g | <0.36 | APHA 4^{th} Edition |
| 2 | E. coli/g | Not Detected | APHA^{h} Edition |

The aforementioned table: 3 provide the information relating to physical parameters (A), color (B), safety parameters (C) and microbiological parameters (D).

The physical parameters (A) will establish the various physical properties of the product of instant invention. In addition, it also discloses the method followed to establish the physical parameters. Further, the color parameters (B) establish that carotenoids are not found and thus the color of the product of instant invention is improved using the process of instant invention. Furthermore, the safety parameters and microbiological parameters establish that the product of instant invention is free from toxicity and adverse effects.

### Example: 7

### Determination of Antihyperglycaemic Activity

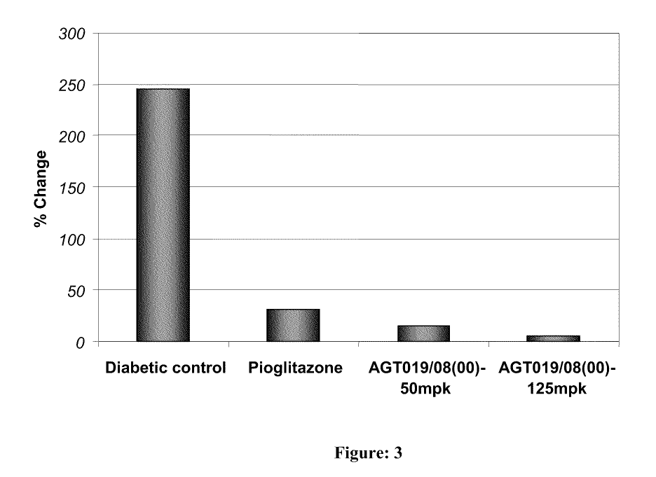

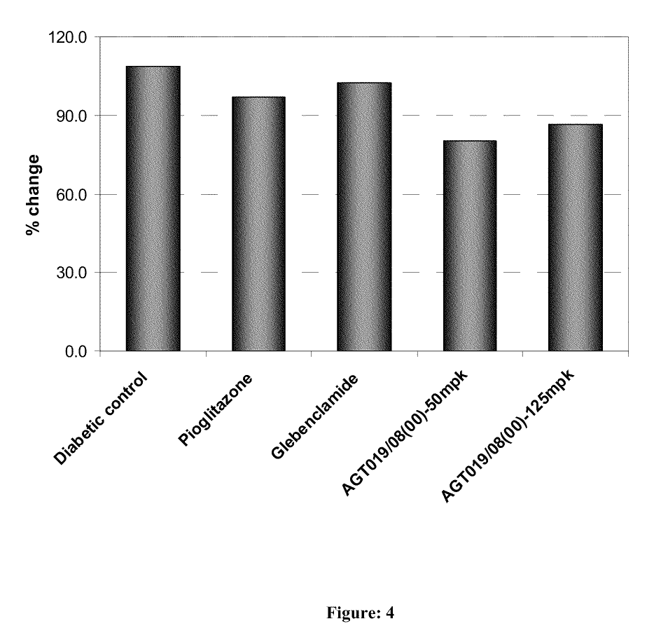

The postprandial blood glucose lowering potential of trigonella water extracts at different concentrations are illustrated in FIG. 3. The diabetic control rat group showed 240% increase in their postprandial blood glucose levels compared to the baseline values of the same group at the start of the study. While the groups treated with pioglitazone, Trigonella water extract at 50, and 125 mpk (milligram per kg body weight) did not show any significant percent change from their baseline values. The FIG. 4, diabetic control rats showed 108.62% rise in the HbA1c level at the end of 6 weeks (42 days) compared to the baseline value at the start of the study (0^{th} Day). The bioactive intervention rats showed 80% rise in HbA1c level at the end of 6 weeks (42 days) compared to the baseline value at 50 mpk and 86.45% at 125 mpk dosage.

1. An organoleptically improved dietary fiber composition obtained from Trigonella foenum-graecum for management of diabetes, said composition comprising proteins 20% w/w to 30% w/w, and galactomannans 32% w/w to 80% w/w optionally along with acceptable additives.

2. The organoleptically improved dietary fiber of claim 1, wherein said composition preferably comprises proteins 25% w/w, and galactomannans 75% w/w.

3. The organoleptically improved dietary fiber of claim 1, wherein said dietary fiber is extracted preferably from the seeds of Trigonella foenum-graecum.

4. The organoleptically improved dietary fiber of claim 1, wherein said organoleptic characters include decolorization, deodorization and debitterization.

5. The organoleptically improved dietary fiber of claim 1, wherein the fiber is a water soluble food supplement.

6. The organoleptically improved dietary fiber of claim 1, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

7. The organoleptically improved dietary fiber of claim 1, wherein said composition is formulated into dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs.

8. A method of preventing and/or managing diabetes in a subject in need thereof, said method comprising the step of administering pharmaceutically acceptable amount of organoleptically improved dietary fiber composition obtained from Trigonella foenum-graecum said composition comprising proteins 20% w/w to 30% w/w, and galactomannans 32% w/w to 80% w/w optionally along with acceptable additives to the subject.

9. The method of preventing and/or managing of claim 8, wherein the subject is an animal or human being.

10. The method of preventing and/or managing of claim 8, wherein the soluble dietary fiber from Trigonella foenum-graecum is administered at dosage ranging between 30 to 125 mg/kg body weight.

11. The method of preventing and/or managing of claim 8, wherein the soluble dietary fiber from Trigonella foenum-graecum is administered preferably at dose of about 50 mg/kg body weight.

12. The method of preventing and/or managing of claim 8, wherein the additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

13. The method of preventing and/or managing of claim 8, wherein the soluble dietary fiber from Trigonella foenum-graecum is formulated into dosage forms selected from a group comprising tablet, troches, lozenges, aqueous or oily suspensions, dispersible powders or granules, emulsion in hard or soft gel capsules, syrups, elixirs, phyotceuticals and neutraceuticals.

14. The method of preventing and/or managing of claim 8, wherein the soluble dietary fiber from Trigonella foenum-graecum is non-toxic and free of adverse effects.

15. A process for extraction of organoleptically improved dietary fiber composition from plant Trigonella foenum-graecum comprising proteins 20% w/w to 30% w/w, and galactomannans 32% w/w to 80% w/w, optionally along with acceptable additives, said process consisting of the steps of:
(a) soaking the plant parts overnight in a first solvent;
(b) powdering plant parts to obtain powder;
(c) extracting the powdered plant parts using a second solvent selected from a group comprising water, hydrochloric acid and combinations thereof;
(d) separating the extracted components;
(e) precipitating the separated components to obtain gum; and
(f) washing precipitated gum followed by drying to obtain organoleptically improved dietary fiber.

16. The process of claim 15, wherein the plant parts preferably seeds were soaked in water over night followed by draining the brown colored water 3 to 4 times.

17. The process of claim 15, wherein said dietary fiber is milky white in color, tasteless and flavorless.

18. The process of claim 15, wherein the plant parts are selected form a group comprising root, shoot, leaf and seeds or the whole plant.

19. The process of claim 15, wherein the preferred plant parts are seeds.

20. The process of claim 15, wherein the plant parts are powdered either manually or mechanically.

21. The process of claim 15, wherein the first solvent and the second solvent are independently selected from a group comprising water, concentrated hydrochloric acid and combinations thereof.

22. The process of claim 15, wherein the first solvent and the second solvent are a combination of water and hydrochloric acid.

23. The process of claim 22, wherein the water to acid ratio is ranging from 1:100 to 1:1000.

24. The process of claim 23, wherein the water to acid ratio is preferably 1:200.

25. The process of claim 15, wherein the extraction is carried out for a time period ranging from 3 to 16 hrs.

26. The process of claim 25, wherein the extraction is carried out for a time period preferably about 4 hrs.

27. The process of claim 15, wherein said extracted components are separated by a method selected from a group comprising centrifugation, filtration and sedimentation.

28. The process of claim 15, wherein said precipitation is achieved using organic solvents selected from a group comprising methanol, ethanol, anhydrous ethanol, propanol, butanol, ethyl acetate and combinations thereof.

29. The process of claim 15, wherein said precipitation is achieved preferably using solvents ethyl alcohol and ethyl acetate at a ratio of 2:1.

30. The process of claim 15, wherein said precipitation results in obtaining a gum as heavier phase.

31. The process of claim 15, wherein said gum is washed using a solvent selected from a group comprising water, methanol, ethanol, anhydrous ethanol, propanol, butanol and combinations thereof.

32. The process of claim 31, wherein said gum is washed using hydro alcoholic mixture at a concentration ranging from 80% to 95%.

33. The process of claim 32, wherein said gum is washed using anhydrous alcohol at a concentration preferably about 95%.

34. The process of claim 32, wherein said gum is washed for a time period ranging from 30 to 90 minutes.

35. The process of claim 32, wherein said gum is washed for a time period preferably about 35-45 minutes.

36. The process of claim 15, wherein the gum is dried by freeze drying method at a temperature preferably about −70° C.

37. The process of claim 15, wherein the dietary fiber is obtained from Trigonella foenum-graecum having a yield ranging from 35% to 80%.

38. The process of claim 37, wherein the dietary fiber is obtained from Trigonella foenum-graecum having a yield preferably of about 78±2%.
